(19) United States
(12) Patent Application Publication
(10) Pub. No.: US 2024/0180885 A1
(43) Pub. Date: Jun. 6, 2024

(54) COMPOSITION AND DELIVERY METHOD FOR TREATMENT OF PHARYNGEAL TONSIL

(71) Applicant: Laurence M Shanley, Lakeworth, FL (US)

(72) Inventor: Laurence M Shanley, Lakeworth, FL (US)

(21) Appl. No.: 18/432,624

(22) Filed: Feb. 5, 2024

Related U.S. Application Data
(63) Continuation of application No. 15/153,678, filed on May 12, 2016, now abandoned.

(51) Int. Cl. A61K 31/4425; A61K 9/00; A61K 9/08; A61K 31/045; A61K 36/28; A61K 36/286; A61K 36/31; A61K 36/48; A61K 36/534; A61K 36/54; A61K 36/63; A61K 36/736; A61K 36/87; A61K 36/889; A61K 36/899; A61K 47/10

(52) U.S. Cl. CPC A61K 31/4425; A61K 9/006; A61K 9/08; A61K 31/045; A61K 36/28; A61K 36/286; A61K 36/31; A61K 36/48; A61K 36/534; A61K 36/54; A61K 36/63; A61K 36/736; A61K 36/87; A61K 36/889; A61K 36/899; A61K 47/10

(57) ABSTRACT

A composition of alcohol and essential oils that enable the composition to have a prolonged antimicrobial effect by reducing the normal rate of alcohol dehydration, reducing toxicity, and increasing duration of efficacy with proper application to oral cavity, oropharynx, and directly pharyngeal tonsils. The composition is delivered as a jet stream focusing on the pharyngeal tonsil area.

### CROSS-REFERENCE TO RELATED APPLICATIONS

The present invention is a continuation of a U.S. patent application Ser. No. 15/153,678, filed on May 12, 2016, which is incorporated herein by reference in its entirety.

### FIELD OF INVENTION

The present invention relates to a composition and a delivery method for the treatment of Infected pharyngeal tonsils, such as sore throat

### BACKGROUND

Sore throats are common. Like colds, the vast majority of sore throats are caused by viral infections, such as a cold or flu. This means most sore throats will not respond to antibiotics. Many people have a mild sore throat at the beginning of every cold. When the nose or sinuses become infected, drainage can run down the back of the throat and irritate it, especially at night. Or the throat itself can be infected.

Some viruses can cause specific types of sore throat. For example, Coxsackievirus sometimes causes blisters in the throat, especially in the late summer and early fall. Mononucleosis and the flu can also cause specific viral throat infections.

Strep throat is the most common bacterial cause of sore throat. Because strep throat can occasionally lead to rheumatic fever, antibiotics are given. Strep throat often includes a fever (greater than 101° F.), white, draining patches on the throat, and swollen or tender lymph glands in the neck. Children may have headaches and stomach pain.

A sore throat is less likely to be strep throat if it is a minor part of a typical cold (with runny nose, stuffy ears, cough, and similar symptoms). Strep cannot be accurately diagnosed by looking at the throat alone. It requires a laboratory test.

Sometimes breathing through the mouth will cause a sore throat in the absence of any infection. During the months of dry winter air, some people will wake up with a sore throat most mornings. This usually disappears after having something to drink. A sore throat may also be caused by something stuck in the throat or allergies (allergic rhinitis).

With a sore throat, sometimes the tonsils or surrounding parts of the throat are inflamed. Either way, removing the tonsils to try to prevent future sore throats is not recommended for most children.

It has been observed that infections start from pharyngeal tonsil and further spread to lungs. So, getting rid of the infection at the early stage can prevent the spread of infection into lungs.

A need is therefore appreciated for a novel composition that has broad spectrum antimicrobial properties and targeted delivering said composition.

### SUMMARY OF THE INVENTION

The principal object of the present invention is therefore directed to an antimicrobial composition that has broad spectrum antimicrobial properties at least against bacteria and viruses.

It is another object of the present invention that the composition can be targeted to pharyngeal tonsils.

Still another object of the present invention is that the composition has a smoothening effect.

In one aspect, disclosed is a method for treating throat infections, the method comprising providing an antimicrobial composition consisting of alcohol 20-30% by weight, and oils 70%-80% by weight; and administering the composition as a narrow jet stream directly focused on pharyngeal tonsil. The alcohol is ethanol or isopropyl alcohol. The oils comprise sesame oil.

In one aspect, an elongated nozzle can be used to focus on a region as small as the tonsils. About 75% oil results in desired viscosity of the composition. The nozzle of a bottle used to deliver the disclosed composition can deliver between 0.5-1 ml jet stream focused to the tonsil and throat. The nozzle can rotate, and the length can be extended to about 3 inches to reach the tonsil. The composition can remain in contact for about 5-10 seconds or more, and then can be discarded.

### DETAILED DESCRIPTION

Disclosed is a composition having a broad-spectrum antimicrobial activity against bacteria and viruses. Also, disclosed is a delivery method for targeted delivery of the composition to the pharyngeal tonsil. The composition can be delivered as a jet stream that can be focused on the small area in the mouth, such as the pharyngeal tonsil. The focused jet stream can be advantageous over conventional mist sprays. The conventional mist sprays merely diffuse in mouth spreading all over the mouth but could not effectively deliver a precise dose to the pharyngeal tonsil area. Spreading the spray all over the mouth including tongue is often inconvenient to the user. The present invention overcomes the limitations by directing a focused narrow jet stream of composition in a viscous form. The disclosed viscous composition can be applied to pharyngeal tonsil area in effective concentrations. Moreover, the velocity of the jet stream causes better penetration of the composition into infected tissues. The jet stream can at least replace a liquid layer covering the infected tissues in the posterior mouth i.e., pharyngeal tonsil area by the thick and viscous disclosed composition. Moreover, the viscous composition can form a layer on the infected tissue providing a sustained antimicrobial activity for a longer duration and smoothening of any inflammation present in the infected tissues. The disclosed composition includes viscous oils that have both smoothening and antimicrobial properties.

In a preferred embodiment, 0.5-1 ml jet stream can be delivered targeted to the tonsil area. The viscous oil/alcohol mixture delivered as a direct jet stream to the tonsil area results in a good contact time with the infected tissues and is the key to killing the infection. The disclosed composition can be used according to embodiments of the present invention at an early state of infection, thus, to prevent proliferation to lungs. The disclosed viscous composition, at the site of infection, slowly spreads, in effective concentrations, to treat the infections. Thus, both the viscosity of composition and velocity of jet stream are a critical feature of the invention for enhanced effectiveness. In certain implementations, the dose of composition can be optimized to prevent the spread of composition all over the mouth.

In one implementation, the disclosed composition includes isopropyl or ethyl alcohol for antimicrobial properties. The isopropyl or ethyl alcohol has a wide spectrum antimicrobial effect and is effective against both bacteria and viruses. Moreover, the contact time for antimicrobial effects is also very short i.e., a few seconds. The viscosity of composition and delivery method allows applying the composition to the infected tissues in effective concentrations and provide for more than the required contact time.

In one implementation, the composition may further include a viscous essential oil. The essential oils added provides viscosity to the composition. The essential oil covering the inflamed tissue provides a soothing effect. Moreover, the oils provide for increases contact time of the composition at the site of infection. The essential oils themselves have a broad spectrum of antimicrobial properties. The essential oils enable the composition to have a prolonged antimicrobial effect by reducing the normal rate of alcohol dehydration. Also, the viscous oils used in the disclosed composition prevent any toxicity or undesirable effects of alcohol to mucous membrane of the mouth. The viscosity and proportion of the oils in the composition also play a key role in spreading of the composition over the infected tissues. Thus, the viscosity of oils and proportion of oils in the disclosed composition play a critical role.

In one implementation, the disclosed composition can have ethyl or isopropyl alcohol in a proportion of about 20-30 percent w/w. The oils can be present in a proportion of about 70-80 percent. Preferably, the composition may include alcohol and oils in a proportion of about 25:75 percent w/w.

In certain implementations, the disclosed composition can also be used in dry throat, wherein the composition can have soothing effect as well as preventing action against infection. For example, dry throat caused by environmental conditions, excessive talking, yelling, speaking, singing, medication side effects, and the like.

In a study, the disclosed composition containing alcohol and oils in 25:75 proportions, the composition was found to kill four major bacterial groups (99.9%) that cause upper respiratory infections such as influenza, pneumonia, sore throat, and MRSA in an Invitro study. In another Invitro Corona study also showed a total kill of corona virus which causes common cold, SARS, Covid, etc. The composition was found to be highly effective for reducing tonsil infection. Also, it was established in the studies that the 25% alcohol to oil solution was effective and does not result in any toxicity to the mucous membrane. Moreover, alcohol diluted with oil was found to be much more effective than when diluted with water.

In certain implementations, disclosed is a composition that has disinfectant (mucosal) and moisturizer action that can be applied by direct stream to pharyngeal tonsils to effectively destroy various pathogenic organisms that cause sore throat.

In certain implementation, the composition can include oils or a combination of oils. Examples of such oils include sesame oil, sunflower oil, safflower oil, peanut oil, olive oil, coconut oil, soybean oil, avocado oil, corn oil, almond oil, canola oil, grape oil, black seed oil, oregano oil and the like.

In one implementation, the composition includes an isopropyl alcohol or ethyl alcohol component between 50-70% by weight and 15-35% by total volume. A sesame oil or alternative oil such as sunflower oil, safflower oil, peanut oil, olive oil, coconut oil, soybean oil, avocado oil, corn oil, almond oil, canola oil, grape oil, black seed oil, oregano oil component not exceeding 80% by volume; a lemon oil or lemon balm component not exceeding 4% by volume; a peppermint oil or alternative wintergreen oil, mint oil, spearmint oil, eucalyptus oil, licorice oil, clove oil not exceeding 6%; aloe vera oil extract not exceeding 5%, for supplementing and neutralizing the dehydrating effect, mucosal irritation and toxicity of said alcohol; a proprietary natural and artificial strawberry flavor additive or alternative, cherry, grape, lemon, lime, orange, bubble gum, berry, coffee, vanilla, chocolate, mint, blueberry and does not exceed 8%.

The present invention relates to a throat (mucosal) disinfectant topically applied or by spray that effectively destroys various pathogenic organisms that cause sore throat. The solution coats the throat and specifically the pharyngeal tonsil region and does not rapidly evaporate thus maintaining antimicrobial efficacy for a duration long enough to reduce or even eliminate active infection and the symptoms associated.

The solution is either sprayed or applied to the throat and tonsil area with either a direct jet stream or by swabbing pharyngeal tonsil using friction and a rotating motion. This has shown to have a greater penetrating effect and better antimicrobial efficacy.

The primary active ingredients either isopropyl alcohol or ethyl alcohol have shown to have similar efficacy but by choice the best and most popular results are achieved by utilizing organic ethyl alcohol 50-70% (by weight) and between 15-35% total solution volume.

A proprietary final emulsification process is utilized to make a more soluble solution for packaging and commercialization, but efficacy and antimicrobial nature of product is not disturbed.

A base oil between 45-75% with prioritizing organic and high antiseptic properties like sesame oil or alternative such as sunflower oil, safflower oil, peanut oil, olive oil, coconut oil, soybean oil, avocado oil, corn oil, almond oil, canola oil, grape oil, black seed oil, oregano oil component can be utilized individually or in a combination as long as percentage oil alcohol range falls within minimum and maximum allowable efficacy and dosing standards.

A proprietary variant viscosity flavor strawberry or alternative, cherry, grape, lemon, lime, orange, bubble gum, berry, coffee, vanilla, chocolate, mint, blueberry is added not exceeding 8% depending on desired taste and spray stream for a particular consumer group. For instance, pediatric formulary of the same solution would require less jet stream from spray bottle than adult formulary.

Essential oil peppermint or alternative wintergreen oil, mint oil, spearmint oil, eucalyptus oil, licorice oil, clove oil not exceeding 6% by volume, enhances the antimicrobial effect by cooling the inflamed area tissues and naturally desensitizing pain associated with the infection. Also helps to neutralize the flavor of the alcohol and oils.

Essential oil lemon oil or alternate lemon balm no greater than 4% by volume, is added to enhance antimicrobial efficacy, deliver nutrients to tissues, and helps to neutralize the after taste of alcohol and oils.

Essential oil aloe vera extract no greater than 4% by volume, is added to enhance tissue healing and regeneration.

The solution has been shown to reduce or eliminate the infection in the throat better than other solutions including, mouthwash, saltwater solution, over the counter throat sprays and peroxide base products.

The solution has been shown to reduce or eliminate germs that cause bad breath, the oil base helps keep the solution on the tongue and pharyngeal mucosa for longer duration.

The solution has shown to kill common respiratory bacteria in time kill studies (99.9% effective). Alcohol products for the throat have never been presented with this activity because of rapid evaporation or dilution with saliva.

The solution has shown to kill respiratory virus in time kill studies 98.33-99.9%. Alcohol products for the throat have never demonstrated this type of efficacy for virus because of rapid evaporation or dilution, such as Rhinovirus and Adenovirus. These viruses are different in structure and are harder to penetrate. These viruses are classified as non-enveloped vims. Usually, alcohol does not maintain itself on any surface to effectively penetrate and destroy these microorganisms, this is due to rapid evaporation and dilution of saliva. To effectively destroy these viruses, it requires prolonged contact with an antimicrobial. Solutions such as iodine, chlorine and peroxide can breakdown and destroy these vims but it not suitable for application on a mucosal surface.

The solution even in the highest dosage formulary has shown to not produce dermal irritation or dermal toxicity to mucosal tissues. The alcohol is diluted by up to thirty percent with water and then that mixture is diluted by up to 85% with oil. No adverse side effects have ever been reported during clinical testing.

Time Kill Studies

| | |
|---|---|
| Rhinovirus (Common Cold) | 98.33% |
| Coronavirus (SARS) | 99.99% |
| Influenza A (Bird Flu) | 99.99% |
| Adenovirus | 99.99% |
| Staphylococcus Aureus | 99.99% |
| Streptococcus Pneumoniae | 99.99% |
| Streptococcus Pyogenes | 99.99% |
| Hemophilius Influenzae | 99.99% |

SKIN IRRITATION STUDY: Under the conditions of the study, irritation was observed on the skin of the rabbits. The primary irritation index was calculated to be 1.08. The test article would not be considered a primary irritant to the skin since the empirical score was less than 5.00. It has been determined by the FDA that a score below 5.00 on rabbits is very safe for human skin.

DERMAL TOXICITY STUDY: Under the conditions of the study, there was no mortality or significant evidence of toxicity observed in the rabbits. The test article would not be considered toxic at a dose of 2 g/kg by the dermal route in the rabbits.

In one implementation, disclosed is an antimicrobial composition of mixed component for topical application in the throat, comprising: an ethyl or isopropyl alcohol component between 15%-35% by weight and 50%-70% by volume; a composition of various base and essential oils between 60%-80% by weight; a proprietary flavor additive no greater than 5% by weight for supplementing, neutralizing, activating and reducing the dehydrating effect of said alcohol thus enabling the necessary contact time effectively kill the germs that cause sore throat without mucosal toxicity or irritation. The peppermint, spearmint, menthol or like essential oil no greater than 4% by weight, in combination with solution, neutralizes the undesirable affect from alcohol aroma, temporarily reduces associated pain from a throat infection, temporarily improves sensation of breathing and provides temporary flavor enhancement. The composition is created with a high viscosity and is applied with direct high velocity or friction in order to penetrate affected or targeted tissues resulting in a greater antimicrobial effect. Essential oils are added for increasing natural antimicrobial effect. A proprietary emulsification process may be utilized as an alternative to manual mixing. Said base oils between 60% and 80% by weight such as sunflower oil, safflower oil, peanut oil, olive oil, coconut oil, soybean oil, avocado oil, corn oil, almond oil, canola oil, grape oil black seed oil, oregano oil component can be utilized individually or in a combination and have shown to have natural antiseptic, absorption, and tissue regeneration properties.

Said proprietary variant viscosity flavor additives such as strawberry or alternative, cherry, grape, lemon, lime, orange, bubble gum, berry, coffee, vanilla, chocolate, mint, blueberry not exceeding 8% by weight is utilized, which enhances or aids in neutralizing the alcohol affect or aroma. Said essential lemon oil or alternate lemon balm no greater than 4% by volume, may be added to enhance antimicrobial efficacy, deliver nutrients to tissues, and helps to neutralize the after taste of alcohol and oils. said essential aloe vera oil extract no greater than 4% by volume, is added to enhance tissue healing and regeneration. Antimicrobial solutions have been shown to reduce or eliminate the infection in the throat better than other solutions including, mouthwash, saltwater solution, over the counter throat sprays and peroxide base products. The said antimicrobial solution has shown to reduce or eliminate germs that cause bad breath, the oil base helps reduce dehydration of active ingredients on the tongue and pharyngeal mucosa. The said composition has shown to kill common respiratory bacteria in time kill studies (99.9% effective). Alcohol products for the throat have never been presented with this activity because of rapid evaporation or dilution with saliva. The said composition has shown to kill respiratory virus in time kill studies 98.33-99.9%. Alcohol products for the throat have never demonstrated this type of efficacy for virus because of rapid evaporation or dilution. The said composition even in the highest dosage formulary has shown to not produce dermal irritation or dermal toxicity to mucosal tissues. The alcohol is diluted by up to thirty percent with water and then that mixture is diluted by up to 85% with oil. No adverse side effects have ever been reported during clinical testing.

What is claimed is:

1. A method for treating throat infections, the method comprising:
providing an antimicrobial composition consisting of:
alcohol 20-30% by weight, and
oils 70%-80% by weight; and
administering the composition as a narrow jet stream directly focused on pharyngeal tonsil.

2. The method of claim 1, wherein the alcohol is ethanol.

3. The method of claim 1, wherein the alcohol is isopropyl alcohol.

4. The method of claim 1, wherein the oils comprise sesame oil.
